(19) United States
(12) Patent Application Publication
(10) Pub. No.: US 2025/0319061 A1
(43) Pub. Date: Oct. 16, 2025

(54) COMBINATION TREATMENT FOR ADENOCARCINOMA

(71) Applicant: The General Hospital Corporation, Boston, MA (US)

(72) Inventors: David T. Ting, Dover, MA (US); Eunae You, Boston, MA (US); Richard Y. Ebright, Boston, MA (US); Niyati Desai, North Quincy, MA (US)

(21) Appl. No.: 18/868,614

(22) PCT Filed: May 25, 2023
(86) PCT No.: PCT/US2023/023492

§ 371 Date: Nov. 22, 2024

Related U.S. Application Data
(60) Provisional application No. 63/345,657, filed on May 25, 2022.

(51) Int. Cl. A61K 31/365; A61K 31/4745; A61K 31/513; A61K 31/519; A61K 31/555; A61P 35/00

(52) U.S. Cl. CPC A61K 31/365; A61K 31/4745; A61K 31/513; A61K 31/519; A61K 31/555; A61P 35/00

(57) ABSTRACT

Described herein are methods for treating a carcinoma in a subject, the method comprising administering to the subject a therapeutically effective amount of (5z)-7-oxozeaenol (Oxo) or an analog thereof, optionally in combination with chemotherapy.

### CLAIM OF PRIORITY

This application claims the benefit of U.S. Provisional Application Ser. No. 63/345,657, filed on May 25, 2022. The entire contents of the foregoing are incorporated herein by reference.

### TECHNICAL FIELD

Described herein are methods for treating a carcinoma in a subject, the method comprising administering to the subject a therapeutically effective amount of (5z)-7-oxozeaenol (Oxo) or an analog thereof, optionally in combination with chemotherapy.

### BACKGROUND

Drug resistance remains problematic in cancer therapy. Epithelial-to-mesenchymal transition (EMT) has been identified as a major factor in development of drug therapy resistance in a number of adenocarcinomas including Pancreatic ductal adenocarcinoma (PDAC) (De Las Rivas et al., Archives of Toxicology volume 95, pages2279-2297 (2021); Zeng et al., Int J Mol Sci. 2019 September; 20(18): 4504).

### SUMMARY

Provided herein are methods for treating a carcinoma in a subject, the method comprising administering to the subject a therapeutically effective amount of (5z)-7-oxozeaenol (Oxo) or an analog thereof, optionally in combination with chemotherapy.

Also provided herein are compositions comprising (5z)-7-oxozeaenol (Oxo) or an analog thereof for use in a method of treating a carcinoma in a subject, optionally wherein the method further comprises administering chemotherapy to the subject in combination with Oxo.

In some embodiments, the carcinoma is an adenocarcinoma, e.g., pancreatic adenocarcinoma or colorectal adenocarcinoma. In some embodiments, the carcinoma is pancreatic adenocarcinoma.

In some embodiments, the Oxo or analog thereof is administered in a nanoparticle.

In some embodiments, the method comprises administering Oxo. In some embodiments, the method comprises administering E6201 (1H-2-Benzoxacyclotetradecin-1,7(8H)-dione, 14-(ethylamino)-3,4,9,10-tetrahydro-8,9,16-trihydroxy-3,4-dimethyl-, (3S,4R,5Z,8S,9S,11E)-); L-783,277; hypothemycin; ER-803064; or an exo-enone analogue of (5Z)-7-oxozeaenol.

In some embodiments, the chemotherapy comprises administering one, two, three, or all four of: leucovorin calcium (folinic acid), fluorouracil, irinotecan hydrochloride, and oxaliplatin. In some embodiments, the chemotherapy comprises administering a FOLFIRINOX, FOLFOX, FOLFIRI, or FOLFOXIRI chemotherapy regimen. In some embodiments, the chemotherapy is not or does not comprise administering doxorubicin.

In some embodiments, the subject does not have multiple myeloma, cervical, ovarian, gastric, breast, or colon/colorectal cancer.

Additionally provided herein are methods for treating a carcinoma in a subject, comprising administering to the subject a therapeutically effective amount of (5z)-7-oxozeaenol (Oxo) or an analog thereof, in combination with chemotherapy comprising administering a FOLFIRINOX, FOLFOX, FOLFIRI, or FOLFOXIRI chemotherapy regimen. Also provided are compositions comprising (5z)-7-oxozeaenol (Oxo) or an analog thereof for use in a method of treating a carcinoma in a subject, the method comprising administering to the subject a therapeutically effective amount of Oxo or an analog thereof in combination with chemotherapy comprising administering a FOLFIRINOX, FOLFOX, FOLFIRI, or FOLFOXIRI chemotherapy regimen.

In some embodiments, the carcinoma is an adenocarcinoma, e.g., pancreatic adenocarcinoma or colorectal adenocarcinoma. In some embodiments, the carcinoma is pancreatic adenocarcinoma. In some embodiments, the subject does not have multiple myeloma, cervical, ovarian, gastric, breast, or colon/colorectal cancer. In some embodiments, the chemotherapy is not or does not comprise administering doxorubicin.

Provided herein are methods of treating a cancer in a patient by administering to the patient an effective amount of 5z-7-Oxozeaenol. In some embodiments, the patient is treated with FOLFIRINOX either simultaneously or after treatment with 5z-7-Oxozeaenol. In some embodiments, the cancer is an epithelial cell cancer, e.g., a pancreatic cancer.

Also provided herein are methods of enhancing the effectiveness of chemotherapy in a patient comprising administering to the patient 5z-7-Oxoeaenol in an amount sufficient to enhance the effectiveness of the chemotherapy. In some embodiments, the chemotherapy is FOLFIRINOX. In some embodiments, the 5z-7-Oxoeaenol is administered prior to or at the same time as the chemotherapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Characterization of Patient-Derived PDAC Cell Lines. (a) Heatmap showing the epithelial (E), exocrine (Exo) and quasimesenchymal (QM) genes of patient-derived PDACs. (b-c) QM (b) and E (c) PDACs were seeded on 96-well round bottom ultra-low attachment (ULA) plates, then incubated with 1X FFX for 7 days. Graphs showing relative cell viability measured using CellTiter Glo assay.

FIGS. 2A-D. PDAC Cell Lines Enriched to E State Following (5z)-7-Oxozeaenol Treatment. (a) Images showing RNA-ISH results for E and QM signatures in PDAC cell lines. (b) Pie charts with quantifications of E/QM signatures after incubation with Oxo (10 μM) for 10 days. (c) PDAC3, 9 and 6 cells were treated with Oxo (10 μM) for 10 days in tumorsphere (3D) condition. Heatmap showing the expression levels of the E/QM signatures. (d) Comparison of normalized counts of genes in the Hallmark EMT geneset between cells treated with or without 5z-7-oxozeaenol, demonstrating significantly decreased mesenchymal gene expression in PDAC 3, 6, and 8 after 5z-7-oxozeaenol treatment, and numerically decreased mesenchymal gene expression in PDAC 9 after 5z-7-oxozeaenol treatment.

FIGS. 3A-D. Graphs showing relative cell growth of (a) QM and (b) E PDACs in 3D condition upon FFX and/or Oxo treatment with indicated concentrations for 2 weeks. Cell viability was measured using CellTiter Glo assay. Images showing growing PDACs (in 3D) under these conditions. (c) Graphs with results for clonogenic assay upon FFX and/or Oxo treatment with indicated concentrations for 2 weeks. Then, cells were fixed with 3.7% PFA and stained with crystal violet. Relative colony area was measured using Image J. (d) Images with 2D (Boyden chamber) and 3D invasion assays (type I collagen embedded) of PDAC cell lines incubated with Oxo for 3 days.

FIGS. 4A-F. Effects of TAK1 Knockout in PDAC EMT Plasticity. (a) Western blots showing complete ablation of TAK1 across PDAC cell lines. (b) Heatmaps based on RNA-ISH and total RNA-seq showing EMT signatures in TAK1 KO PDAC6 and PDAC9 cell lines. (c) Clonogenic assays for WT (non-targeting single guide RNA, sgRNA) and TAK1 KO cell lines treated with FFX for 2 weeks. Colony area was determined by Image J. (d) Images with 2D invasion assay (Boyden chamber) in TAK1 KO PDAC cell lines. (e) Graphs confirming functional ablation of TAK1 represented by TAK1 downstream activity of p-MEK1/2 and p-ERK1/2 under TGFbeta stimulation. (f) Graphs with results for clonogenic assay upon FFX and/or Takinib treatment with indicated concentrations for 2 weeks.

FIGS. 5A-D. Alternative Pathways Involving PDAC EMT Plasticity. (a) Phospho-proteomics experimental design scheme. (b) Volcano plots showing up- and down-regulated phosphosites in QM and E PDACs following FFX treatment for 2 weeks. (c) Graphs showing KEGG pathways and biological processes of phosphosites which are up-regulated by FFX in QM PDAC lines. (d) Proposed pathway containing potential target proteins for Oxo.

FIGS. 6A-B. PDAC-CAF co-culture induces resistance to FOLFIRINOX response. (a) Dose response curves in QM and E PDAC monoculture and co-culture with CAF1, as well as CAF1 monoculture, following FFX treatment. (b) FFX IC50 for QM and E PDAC monoculture versus co-culture with CAF1.

FIGS. 7A-C. Treatment with 5z-7-oxozeaenol sensitizes FOLFIRINOX response in PDAC cell lines both alone and co-culture condition. (a) Dose response curves in QM and E PDAC monoculture following combination 5z-7-oxozeaenol and FFX treatment. (b) Dose response curves in QM and E PDAC co-culture with CAF1 following combination 5z-7-oxozeaenol and FFX treatment. (c) Dose response curves in CAF1 monoculture following combination 5z-7-oxozeaenol and FFX treatment.

FIGS. 8A-C. Treatment with Losartan did not affect FOLFIRINOX response in PDAC cell lines both alone and co-culture condition. (a) Dose response curves in QM and E PDAC monoculture following combination losartan and FFX treatment. (b) Dose response curves in QM and E PDAC co-culture with CAF1 following combination losartan and FFX treatment. (c) Dose response curves in CAF1 monoculture following combination losartan and FFX treatment.

FIGS. 9A-E. Combinational drug treatment (Oxo+FFX) in PDAC alone or PDAC+CAF coculture. (a) Experimental schematic of combinatorial drug treatment studies, demonstrating 3 day incubation in 5z-7-oxozeaenol and FFX prior to viability readout. (b) Combination drug treatments of PDAC 3 and PDAC 6, as monoculture or co-culture with CAF1. Values indicate percent killing, as compared to untreated cells, such that 0 indicates no killing and 100 indicates all cells killed. (c) Bliss plots of synergistic activity between 5z-7-oxozeaenol and FFX, where positive values represent synergistic activity, negative values represent anti-synergistic activity, and 0 represents no synergy. Synergy data are based on values from 8B. (d) Combination drug treatments of PDAC 8 and PDAC 9, as monoculture or co-culture with CAF1.

Values indicate percent killing, as compared to untreated cells, such that 0 indicates no killing and 100 indicates all cells killed. (e) Combination drug treatments of CAF1 monoculture. Values indicate percent killing, as compared to untreated cells, such that 0 indicates no killing and 100 indicates all cells killed.

FIG. 10. Combinational drug treatment (Losartan+FFX) in PDAC alone or PDAC+CAF coculture. Combination drug treatments of PDAC 3 and PDAC 6, as monoculture or co-culture with CAF1. Values indicate percent killing, as compared to untreated cells, such that 0 indicates no killing and 100 indicates all cells killed.

### DETAILED DESCRIPTION

Transcriptional profiling has defined PDAC into distinct classical epithelial (E) or quasi-mesenchymal (QM) subtypes, and these subtypes exist on a continuum of interconverting cell states. This epithelial to mesenchymal transition (EMT) plasticity is thought to be important for metastatic dissemination and intrinsic resistance to chemotherapy.

TAK1 has been identified as an important driver of EMT in mouse circulating tumor cells, and (5z)-7-oxozeaenol (Oxo) was identified as a small molecule inhibitor of TAK1 (Yu et al., Nature. 2012 Jul. 26; 487(7408): 510-513). As shown herein, in human patient derived PDAC cell lines Oxo is an EMT plasticity inhibitor, and administration of Oxo or a derivative thereof inhibits development of resistance to chemotherapy, e.g., treatment with 5-fluorouracil/leucovorin with irinotecan and oxaliplatin (FOLFIRINOX).

### Methods of Treatment

Described herein are methods for the treatment of cancer, including solid tumors, e.g., carcinomas, e.g., adenocarcinomas. Generally, the methods include administering a therapeutically effective amount of a treatment comprising Oxo or an analog thereof, and optionally a chemotherapy, e.g., FOLFIRINOX regimen, to a subject who is in need of, or who has been determined to be in need of, such treatment. In some embodiments, the Oxo is administered before, during, or after administration of one or more doses of the chemotherapy, e.g., before or after the subject begins to show resistance to the chemotherapy. The methods can also include radiotherapy, immunotherapy (e.g., with an immune checkpoint inhibitor) and/or resection.

As used in this context, to “treat” means to ameliorate at least one symptom of the cancer. For example, a treatment can result in a reduction in tumor size or growth rate. Administration of a therapeutically effective amount of a compound described herein for the treatment of a cancer will result in a reduction in tumor size or decreased growth rate, a reduction in risk or frequency of reoccurrence, a delay in reoccurrence, a reduction in metastasis, a reduction in risk or frequency of development of treatment resistance, a delay in development of treatment resistance, increased survival, and/or decreased morbidity and mortality, inter alia.

The term “carcinoma” is art recognized and refers to malignancies of epithelial or endocrine tissues including respiratory system, gastrointestinal system, genitourinary system, testicles, breasts, prostate, endocrine system, and melanomas. In some embodiments, the disorder is a solid tumor, e.g., breast, prostate, pancreatic, brain, gastric, hepatic, lung, kidney, skin, or colorectal cancer. In some embodiments, the cancer is an “adenocarcinoma,” i.e., a carcinoma derived from glandular tissue or in which the tumor cells form recognizable glandular structures, including breast, prostate, pancreas, esophagus, colon/rectum, stomach, and lung cancers (e.g., non-small cell lung cancer). In some embodiments, the cancer is not multiple myeloma, cervical, ovarian, gastric, breast, or colon/colorectal cancer.

Methods for identifying subjects as having cancer are known in the art, and can include blood work and imaging studies, as well as biopsy (see, e.g., Mullangi and Lekkala, Adenocarcinoma. [Updated 2022 Sep 26]. In: StatPearls [Internet]. Treasure Island (FL): StatPearls Publishing; 2023 Jan-. Available from ncbi.nlm.nih.gov/books/NBK562137/).

An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day, or once every other week (e.g., every 14 days). The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

(5z)-7-Oxozeaenol (Oxo) and Analogs

The present methods include administering (5z)-7-oxozeaenol (Oxo) or an analog thereof.

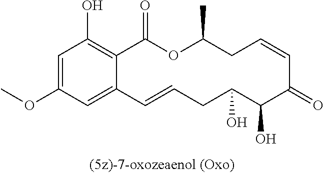 [SMILES: COc1cc(O)c2c(c1)/C=C/C[C@H](O)[C@H](O)C(=O)/C=C\C[C@H](C)OC2=O]

Oxo is a fungal metabolite that binds ATP and has been shown to inhibit a number of kinases including TAK1, MEK, and FLT3.

Analogs include macrocyclic compounds, e.g., as described in WO 2003/076424, e.g., comprising formula (I):

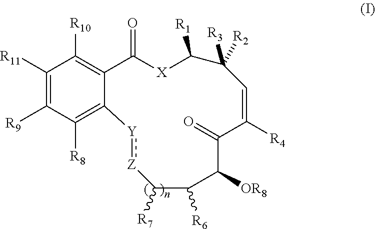

wherein R₁ is hydrogen, aliphatic, heteroaliphatic, alicyclic, heteroalicyclic, aryl or heteroaryl;
R₂ and R₃ are each independently hydrogen, halogen, hydroxyl, protected hydroxyl, or an aliphatic, heteroaliphatic, alicyclic, heteroalicyclic, aryl or heteroaryl moiety; or
R₁ and R₂, when taken together, may form a substituted or unsubstituted, saturated or unsaturated cyclic ring of 3 to 8 carbon atoms; or
R₁ and R₃, when taken together, may form a substituted or unsubstituted, saturated or unsaturated cyclic ring of 3 to 8 carbon atoms;
R₄ is hydrogen or halogen;
R₅ is hydrogen, an oxygen protecting group or a prodrug;
R₆ is hydrogen, hydroxyl, or protected hydroxyl;
n is 0-2;
R₇, for each occurrence, is independently hydrogen, hydroxyl, or protected hydroxyl;
R₈ is hydrogen, halogen, hydroxyl, protected hydroxyl, alkyloxy, or an aliphatic moiety optionally substituted with hydroxyl, protected hydroxyl, SR₁₂, or NR₁₂R₁₃;
R₉ is hydrogen, halogen, hydroxyl, protected hydroxyl, OR₁₂, SR₁₂, NR₁₂R₁₃, —X₁(CH₂)ₚX₂-R₁₄, or is lower alkyl optionally substituted with hydroxyl, protected hydroxyl, halogen, amino, protected amino, or —X₁(CH₂)ₚX₂-R₁₄;
wherein R₁₂ and R₁₃ are, independently for each occurrence, hydrogen, aliphatic, heteroaliphatic, alicyclic, heteroalicyclic, aryl or heteroaryl; or a protecting group, or R₁₂ and R₁₃, taken together may form a saturated or unsaturated cyclic ring containing 1 to 4 carbon atoms and 1 to 3 nitrogen or oxygen atoms, and each of R₁₂ and R₁₃ are optionally further substituted with one or more occurrences of hydroxyl, protected hydroxyl, alkyloxy, amino, protected amino, alkylamino, aminoalkyl, or halogen,
wherein X₁ and X₂ are each independently absent, or are oxygen, NH, or —N(alkyl), or wherein X₂-R₁₄ together are N₃ or are a saturated or unsaturated heterocyclic moiety,
p is 2-10, and
R₁₄ is hydrogen, or an aryl, heteroaryl, alkylaryl, or alkylheteroaryl moiety, or is —(C═O)NHR₁₅—(C═O)OR₁₅, or —(C═O)R₁₅, wherein each occurrence of R₁₅ is independently hydrogen, aliphatic, heteroaliphatic, alicyclic, heteroalicyclic, aryl or heteroaryl; or R₁₄ is —SO₂(R₁₆), wherein R₁₆ is an aliphatic moiety, wherein one or more of R₁₄, R₁₅, or R₁₆ are optionally substituted with one or more occurrences of hydroxyl, protected hydroxyl, alkyloxy, amino, protected amino, alkylamino, aminoalkyl, or halogen; or
R₈ and R₉ may, when taken together, form a saturated or unsaturated cyclic ring containing 1 to 4 carbon atoms and 1 to 3 nitrogen or oxygen atoms and is optionally substituted with hydroxyl, protected hydroxyl, alkyloxy, amino, protected amino, alkylamino, aminoalkyl, or halogen;
R₁₀ is hydrogen, hydroxyl, protected hydroxyl, amino, or protected amino; R₁₁ is hydrogen, hydroxyl or protected hydroxyl;
X is absent or is O, NH, N-alkyl, CH₂ or S;
Y is CHR₁₇, O, C═O, CR₁₇ or NR₁₇; and Z is CHR₁₅, O, CO, CR₁₅ or NR₁₅, wherein each occurrence of R₁₇ and R₁₅ is independently hydrogen or aliphatic, or R₁₇ and R₁₅ taken together is —O—, —CH₂— or —NR₁₉—, wherein R₁₉ is hydrogen or lower alkyl, and Y and Z may be connected by a single or double bond; and
pharmaceutically acceptable derivatives thereof.

In some embodiments, the analog is E6201 (1H-2-Benzoxacyclotetradecin-1,7(8H)-dione, 14-(ethylamino)-3,4,9,10-tetrahydro-8,9,16-trihydroxy-3,4-dimethyl-, (3S,4R,5Z,8S,9S,1IE)—, PubChem CID: 10172827):

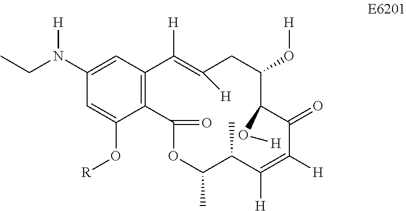

Additional analogs are described in Ellestad et al., Chirality. 2019 Feb;31(2):110-117, including L-783,277, hypothemycin, ER-803064, an exo-enone analogue of (5Z)-7-oxozeaenol, and compounds 11-16 described in Ellestad et al.

Preferably, the analog includes a Z5,6 keto-7enone moiety.

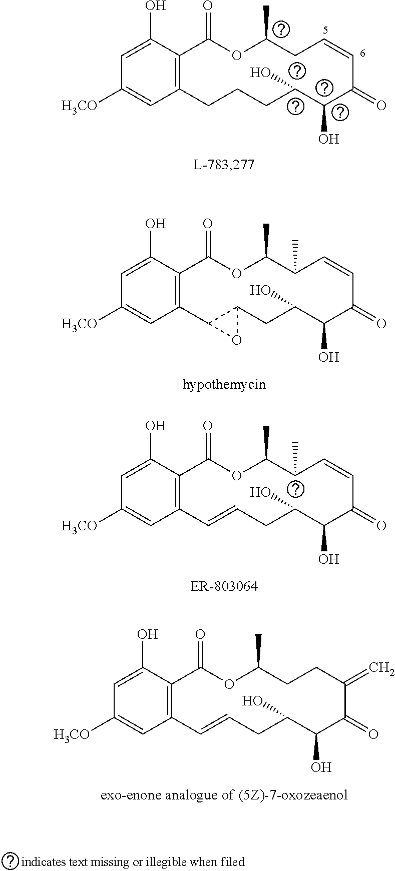

### Chemotherapy

The present methods can include administration of a chemotherapy regimen in combination with Oxo. For example, in some embodiments, e.g., for PDAC or colorectal cancers, the methods can include administering a FOLFOX (leucovorin calcium (folinic acid), fluorouracil, and oxaliplatin); FOLFIRI (infusional fluorouracil, leucovorin, and irinotecan); or FOLFOXIRI regimen (using a lower dose of irinotecan (165 mg/m²), no bolus of 5-FU, and an increase in continuous intravenous 5-FU infusion at 3200 mg/m²; Falcone et al., J Clin Oncol. 2007; 25:1670-1676), or FOLFIRINOX regimen.

FOLFIRINOX is a chemotherapy combination used to treat cancers including pancreatic cancer that includes leucovorin calcium (folinic acid), fluorouracil, irinotecan hydrochloride, and oxaliplatin (see Conroy et al. N Engl J Med 2011; 364:1817; Cercek et al. J Oncol Pract 2016; 12:e459). An exemplary protocol is as follows:

| Drug | Dose | | Administration |
|---|---|---|---|
| Oxaliplatin | 85 | mg/m² | Administer in 500 mL 5% dextrose |
| | | | in water over 85-120 minutes |
| Leucovorin | 400 | mg/m² | Administer in 250 mL 5% dextrose |
| | | | in water over two hours, |
| | | | after oxaliplatin. |
| Irinotecan | 180 | mg/m² | Administer in 500 mL 5% dextrose |
| | | | in water over 90 minutes, concurrent |
| | | | with the last 90 minutes of |
| | | | leucovorin infusion |
| Fluorouracil | 400 | mg/m² | Administer undiluted as a slow IV |
| (FU) bolus | | | push bolus over five minutes |
| | | | immediately after leucovorin. |
| FU | 2400 | mg/m² | Administer in 500 to 1000 mL 0.9% |

normal saline or 5% dextrose in
water as a continuous IV infusion
over 46 hours beginning immediately
after FU IV bolus; alternatively
administer undiluted (50 mg/mL) or
diluted in 100 to 150 mL normal
saline using an ambulatory pump.
Table adapted from FOLFIRINOX Chemotherapy For Metastatic Pancreatic Cancer, Up To Date, 2023, available at uptodate.com/contents/image?imageKey=ONC%2F79571.

Chemotherapy regimens are known in the art and can include administering one or more antimicrotubule agents including polymerizing agents (e.g., taxanes such as paclitaxel or docetaxel) and depolymerizing drugs (e.g., vinca alkaloids such as vincristine, vindesine, vinblastine, or vinorelbine), benzoylphenylureas (BPUs, e.g., NSC-639829), epothilones (e.g., ixabepilone) or estramustine phosphate); and/or one or more platinum-based agents, e.g., cisplatin, oxaliplatin, or carboplatin. In some embodiments, the chemotherapy is not or does not comprise administering doxorubicin.

### Pharmaceutical Compositions and Methods of Administration

The methods described herein include the use of pharmaceutical compositions comprising or consisting of Oxo or an analog thereof as an active ingredient, in combination with administration of the FOLFIRINOX regimen.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Where Oxo or another insoluble analog is used, the composition can be modified to improve solubility for administration. For example, the Oxo can be provided as a stock, e.g., a 10 mg/ml stock, in dimethyl sulfoxide (DMSO), which can be further diluted in an oil, e.g., Peanut Oil (Sigma), (e.g., diluted 10-fold to yield a 1.0 mg/ml stock in 10% DMSO; Kong et al., Immunology. 2015 May; 145(1): 136-149). Alternatives to DMSO for solubilizing Oxo are known in the art, including solvate ionic liquids (SILs) that are equimolar solutions of lithium bistrifluoromethanesulfonimide in either triglyme (G3LiTFSA) or tetraglyme (G4LiTFSA) (Yoganantharajah, et al., BMC Biotechnology volume 18, Article number: 32 (2018); zwitterionic liquid (ZIL) a zwitterion-type ionic liquid containing histidine-like module (Kuroda et al., Communications Chemistry volume 3, Article number: 163 (2020)); and low-molecular weight pentaisomaltose (1 kDa)(Svalgaard et al., Cell Transplant. 2018 September; 27(9): 1407-1412).

Alternatively, the Oxo or analog can be incorporated into or onto a liposome or nanoparticle. For example, gelatin-nanoparticles-encapsulated Oxo have been described (Wang et al., Theranostics. 2022; 12(2): 657-674), as have crosslinked multilamellar liposome vesicles (cMLVs) comprising Oxo (see, e.g., Iriondo et al., Nat Commun. 2018; 9: 1994; Joo et al., Biomaterials. 2013 April; 34(12): 3098-3109).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof. The pharmaceutical compositions can be included in a container, pack, or dispenser together with instructions for administration of Oxo or an analog thereof as part of the FOLFIRINOX regimen. For example, the Oxo or an analog thereof can be administered before, during, or after administration of the FOLFIRINOX regimen, or can be co-administered with one of the drugs in the regimen, or between two of the drugs of the regimen.

### EXAMPLES

The invention is further described in the following examples, which do not limit the scope of the invention described in the claims.

### Example 1. (5z)-7-Oxozeanol as an Epithelial to Mesenchymal Transition (EMT) Plasticity Inhibitor in Pancreatic Ductal Adenocarcinoma (PDAC)

Transcriptional profiling has defined PDAC into distinct classical epithelial (E) or quasi-mesenchymal (QM) subtypes, and these subtypes exist on a continuum of interconverting cell states. This epithelial to mesenchymal transition (EMT) plasticity is thought to be important for metastatic dissemination and intrinsic resistance to chemotherapy. See Porter et al, 2019; Collisson et al., 2011; Kim and Choi, 2012; and Porter et al., 2019.

Our prior work identified TAK1 as an important driver of EMT in mouse circulating tumor cells, and we had identified a compound (5z)-7-oxozeaenol (Oxo) as a small molecule inhibitor of TAK1. Here, we have expanded upon this prior work by evaluating Oxo in human patient derived PDAC cell lines as an EMT plasticity inhibitor.

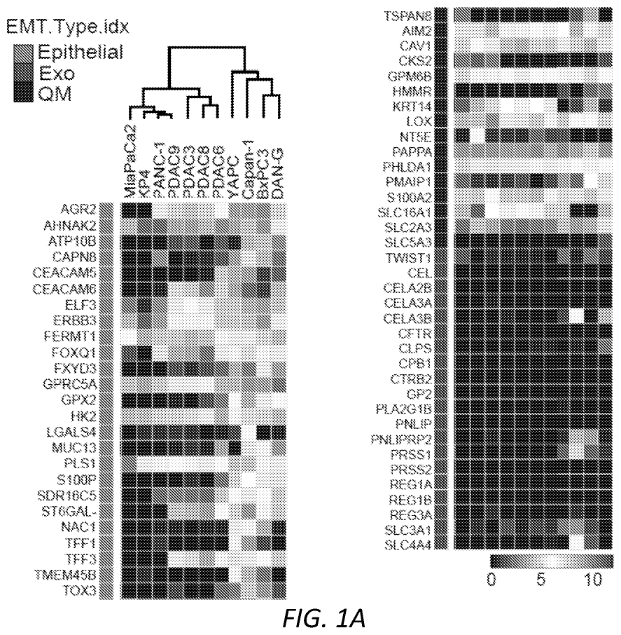

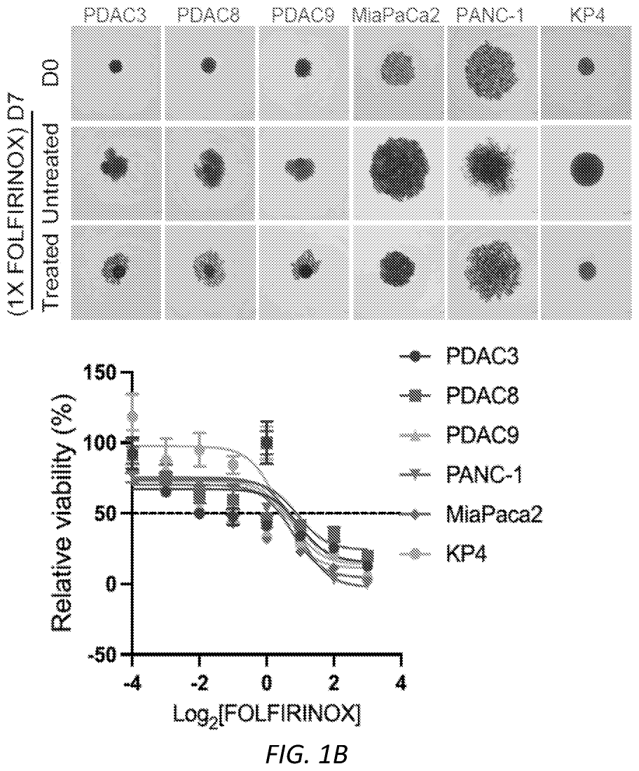

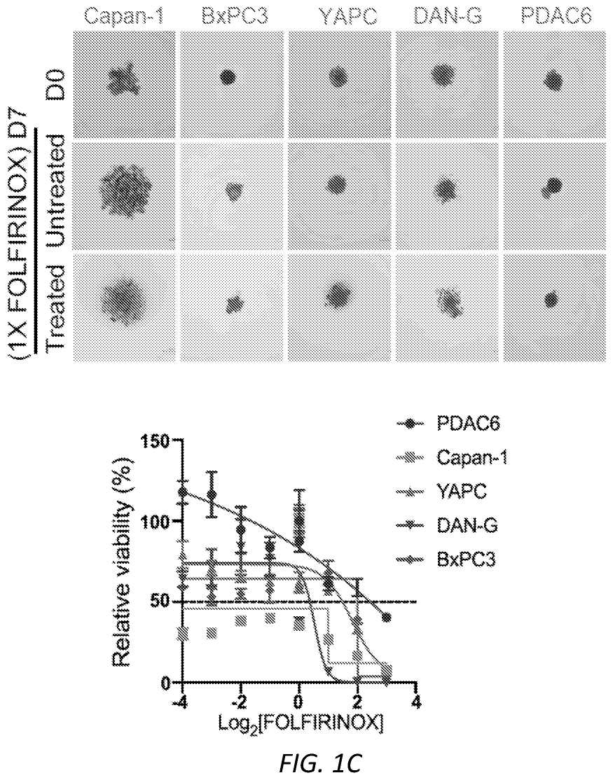

First, we performed total-RNA sequencing to classify PDAC subtypes, and we performed supervised clustering based on genes known to be characteristic of different subtypes (FIG. 1A). The corresponding heatmap demonstrates two major clusters of PDAC cell lines. To examine the anti-cancer drug response depending on PDAC subtypes, we incubated PDAC cell lines with the FOLFIRINOX chemotherapy regimen under 3D tumorsphere conditions (FIG. 1B-C). Cell viability graphs and cell images demonstrate that QM clusters (FIG. 1B) and E clusters (FIG. 1C) were sensitive to FOLFIRINOX.

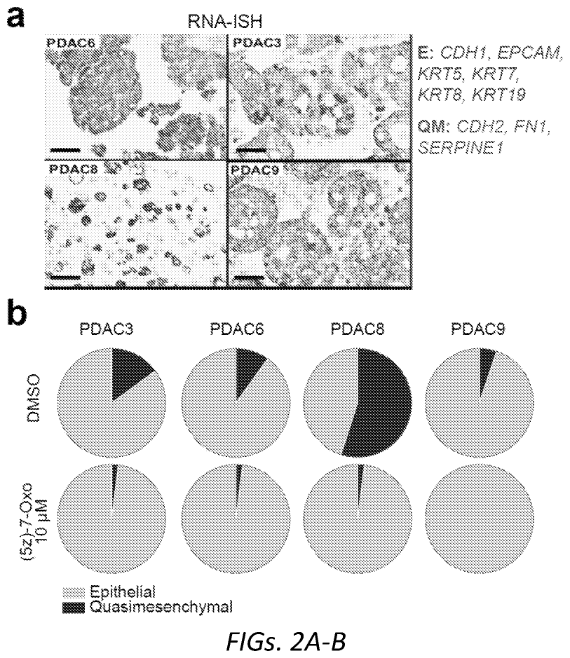

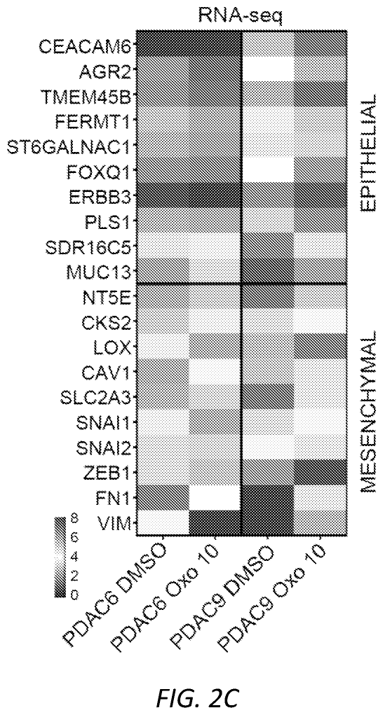

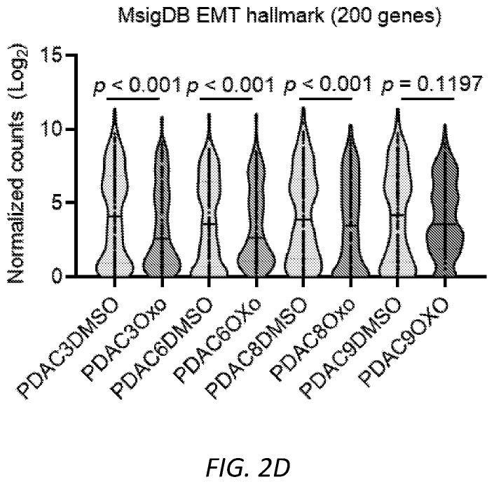

To test if Oxo could block EMT plasticity and improve drug sensitivity in PDACs, we used RNA-ISH, consisting of key E and QM markers, to characterize PDAC cells as E or QM (FIG. 2A). Interestingly, treatment with oxo across PDAC cell lines significantly suppressed QM gene expression and increased E gene expression, both by RNA-ISH (FIG. 2B) and by specific transcripts identified through RNA sequencing (FIG. 2C). In order to assess EMT status after Oxo treatment, we assessed expression of the 200 genes in the Hallmark EMT geneset in PDAC lines with and without Oxo treatment, finding that global mesenchymal gene expression was numerically lower after Oxo treatment in all lines tested, with that difference reaching statistical significance in ¾ of cell lines tested (FIG. 2D). Thus, Oxo treatment globally alters transcriptomic state, leading to downregulation of the mesenchymal state.

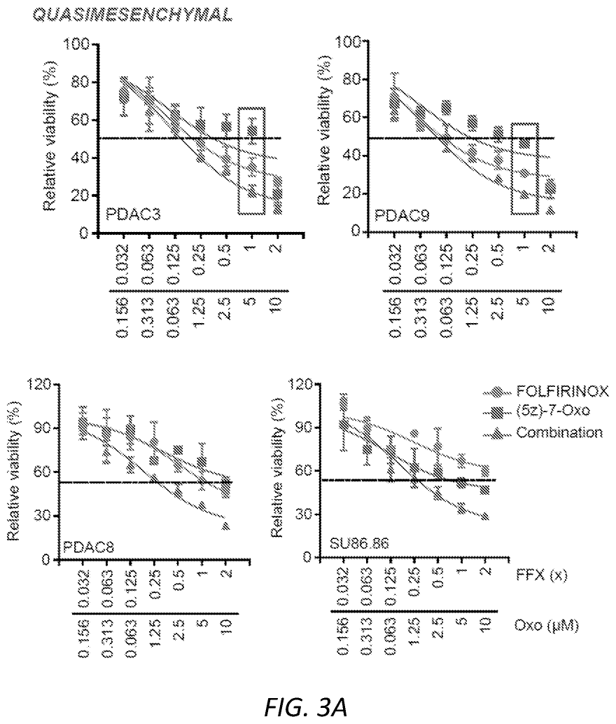

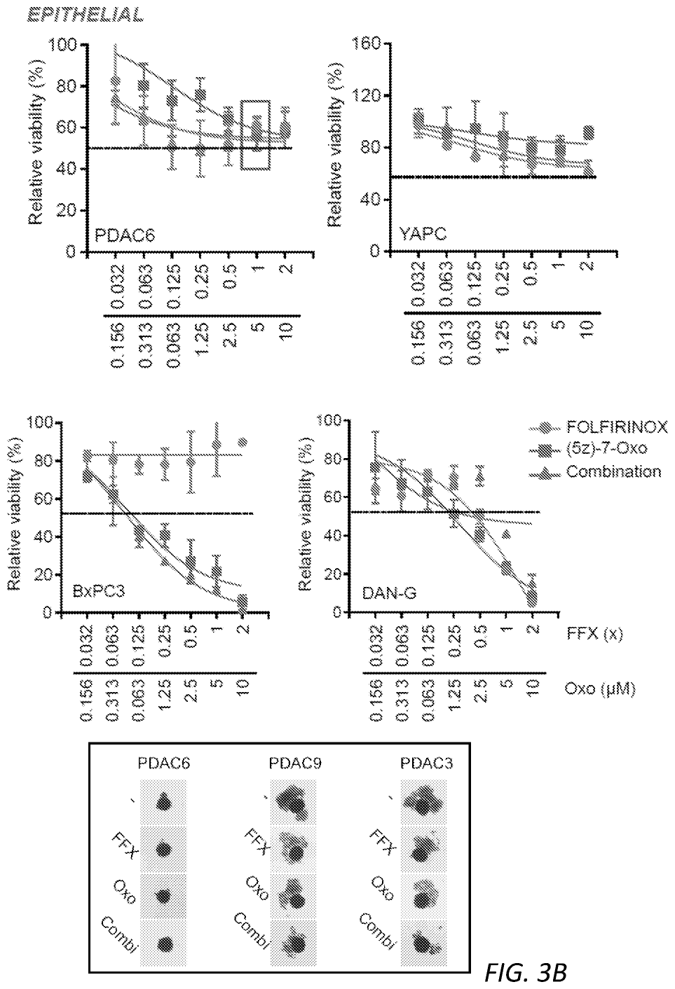

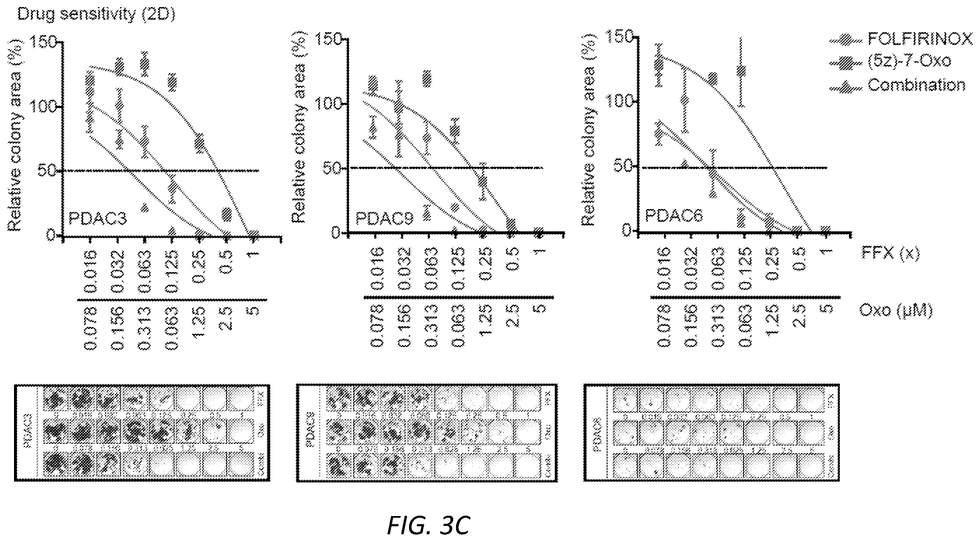

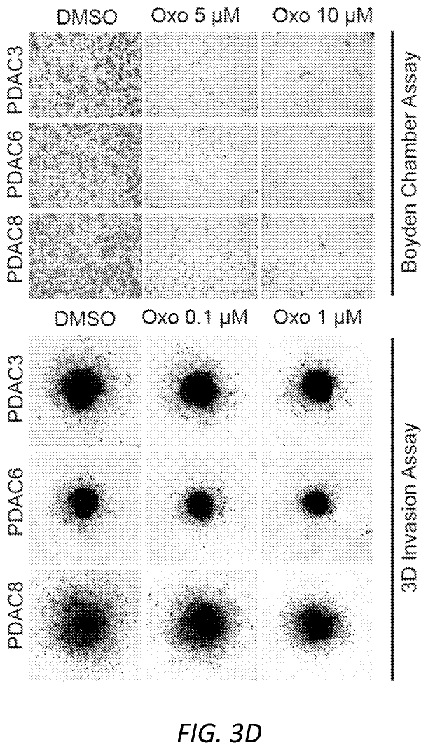

To evaluate the functional consequence of Oxo, Oxo was added to FOLFIRINOX chemotherapy in PDAC cell lines demonstrating a synergistic increased killing of QM cells compared to E cells (FIG. 3A-B). However, we do note some E cells (BxPC3 and DAN-G) are sensitive to Oxo as a single agent. In addition to sensitization to anticancer drug, Oxo-treated PDACs showed a significant reduction in colony formation (FIG. 3C), migratory abilities (FIG. 3D), and invasiveness (FIG. 3D), in line with a reduction in mesenchymal phenotype (FIGS. 2E-F). Thus, Oxo treatment suppresses mesenchymal appearance of cells by both RNA expression markers and functional phenotype.

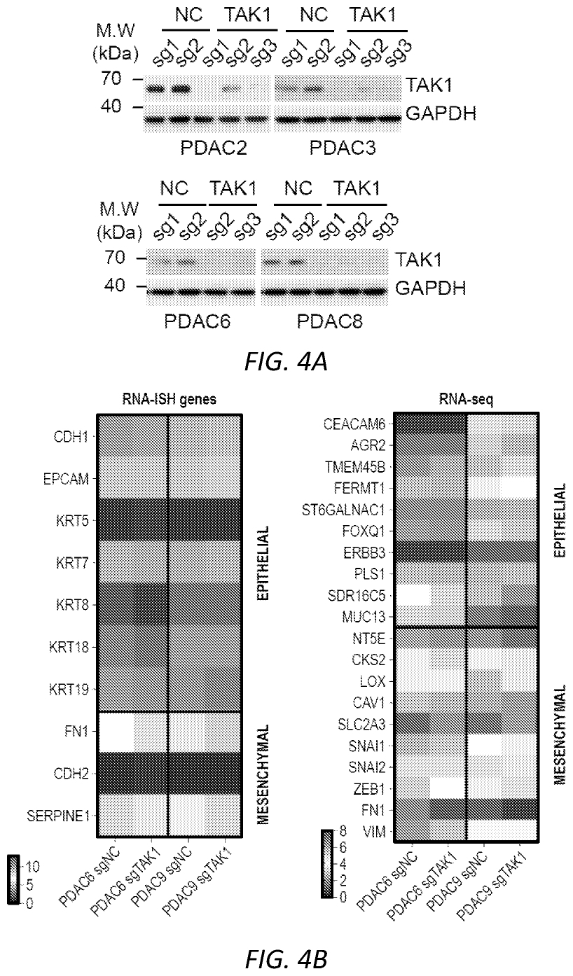

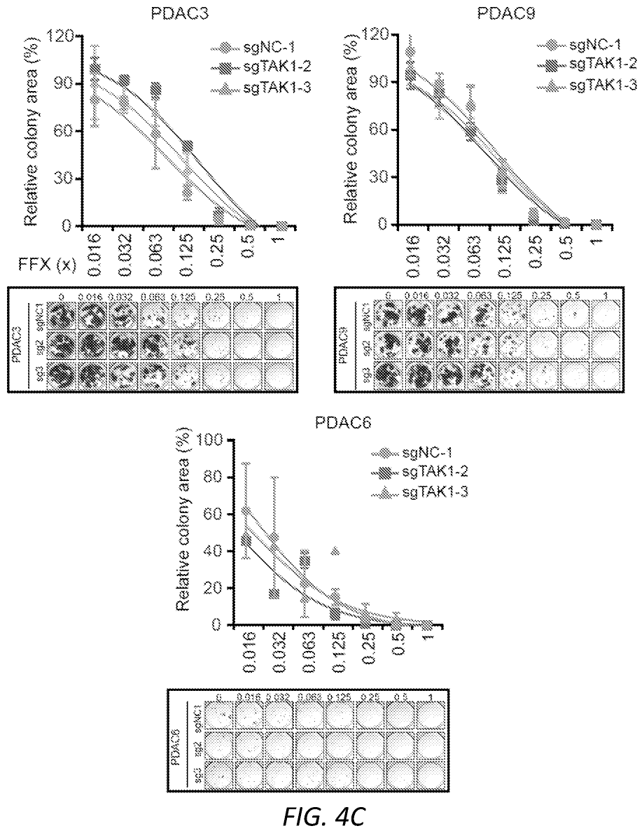

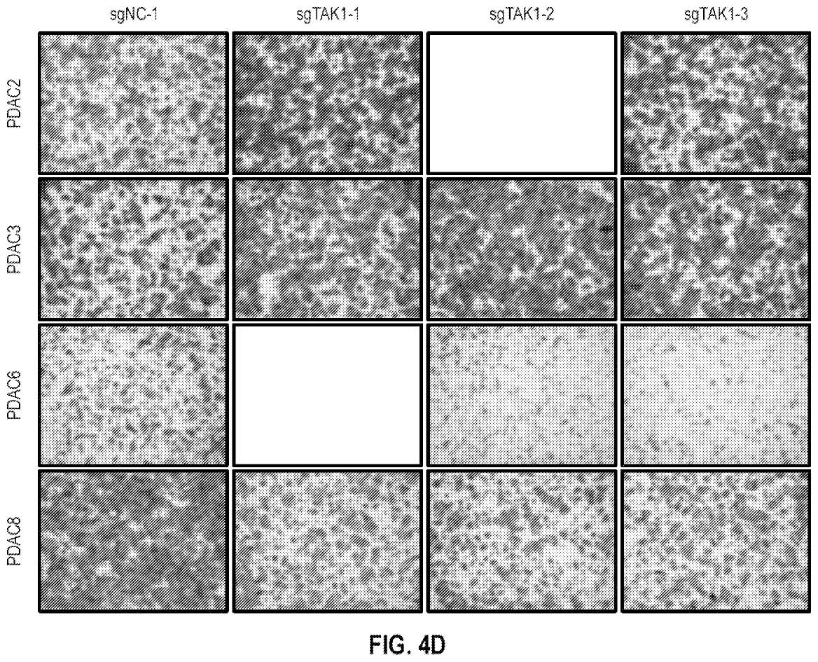

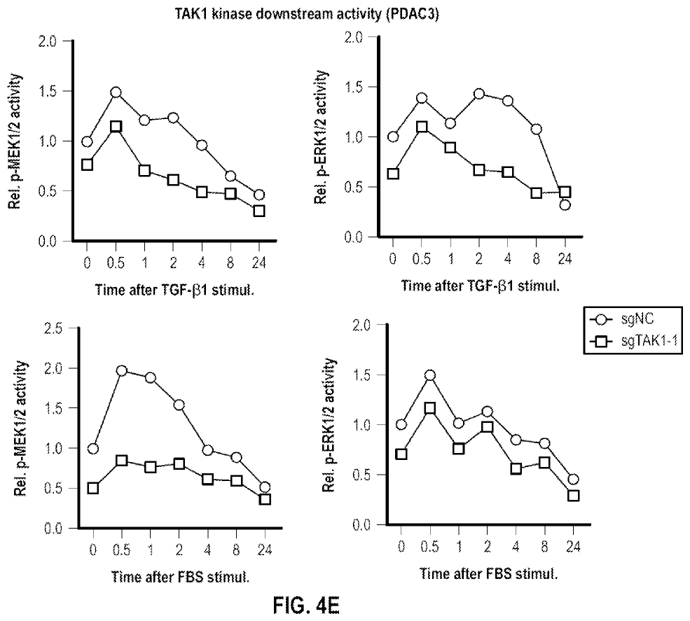

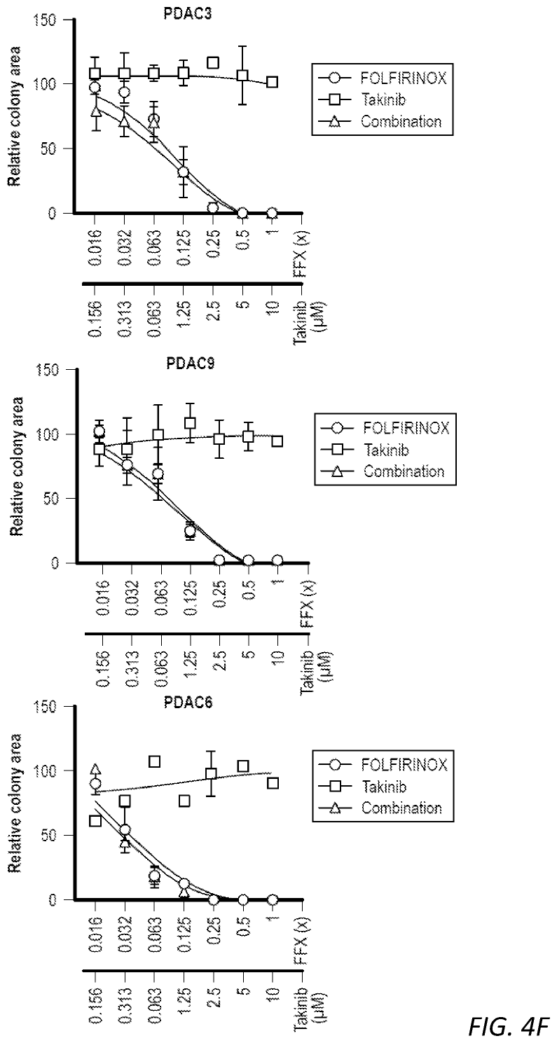

To further demonstrate a potential contribution of TAK1 in the regulation of EMT plasticity, we used CRISPR/Cas9-encoded lentivirus to generate TAK1 knockout cell lines, and complete depletion of TAK1 was confirmed by Western blotting (FIG. 4A). Unexpectedly, we observed knockout of TAK1 did not phenocopy the results of oxo treatment. In contrast, TAK1 knockout showed a slight increase in QM marker genes and had no effect on FOLFIRNOX response (FIGS. 4B-C).

However, TAK1 targeting showed divergent effects on PDAC migration in different cell subtypes, potentially suggesting different response of E vs QM cells to TAK1 loss (FIGS. 4D-E).

Takinib is another selective inhibitor targeting TAK1. Similar to TAK1 KO, Takinib treatment is not able to sensitize FOLFIRINOX response in QM PDAC cells (FIG. 4F). This suggests that QM PDAC cell lines have additional signaling cascades that compensate for TAK1 loss and that the effects of Oxo across different PDAC cell line subtypes are not solely TAK1 dependent.

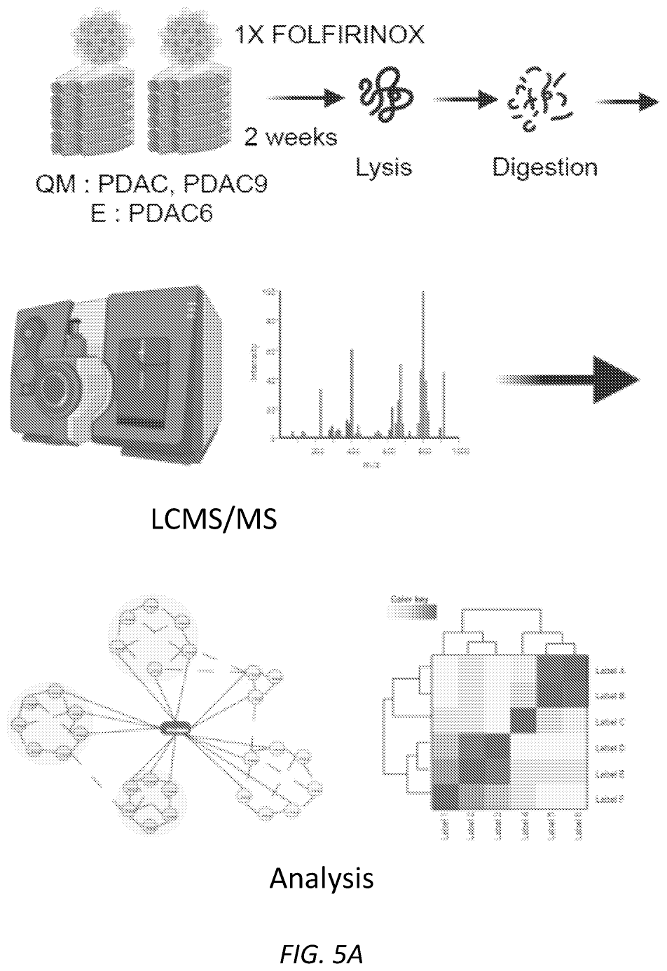

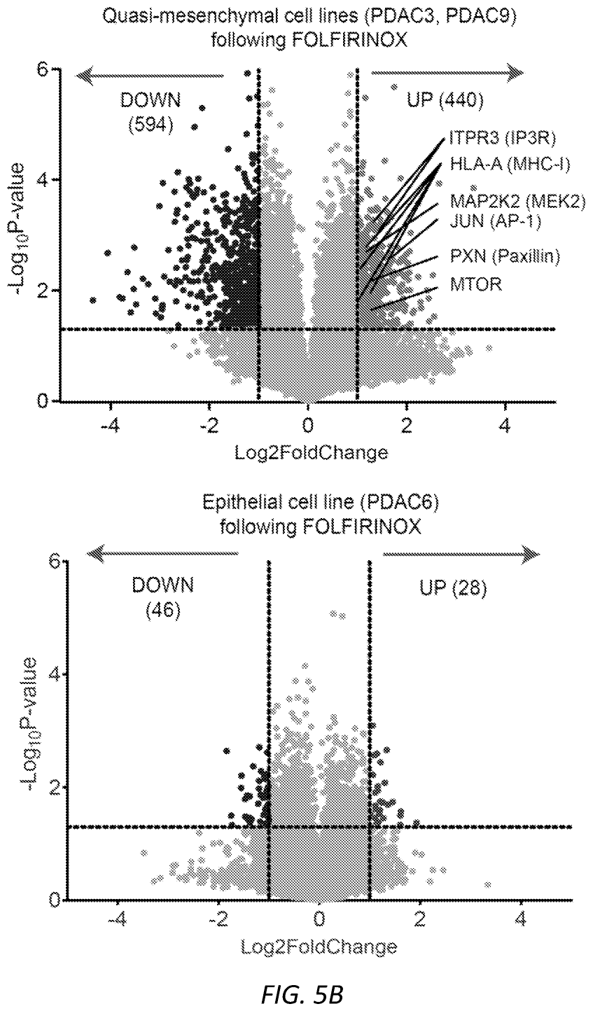

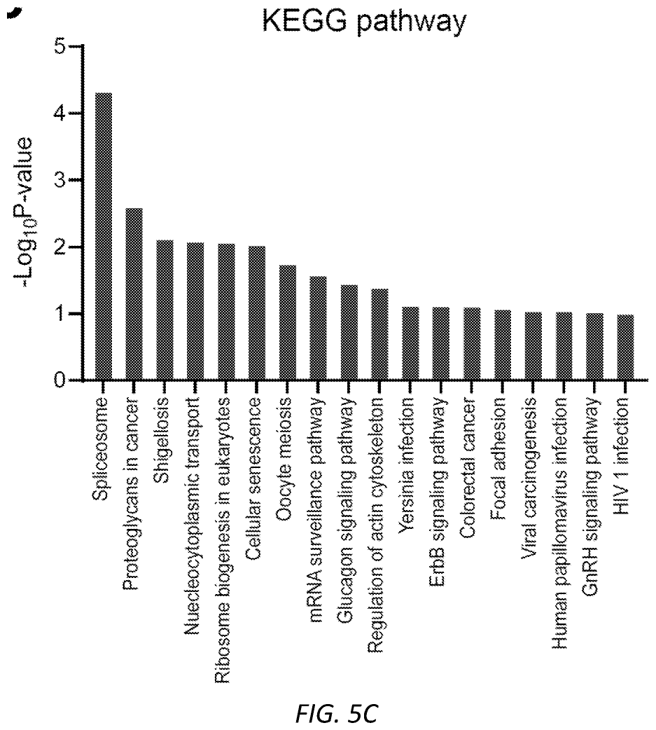

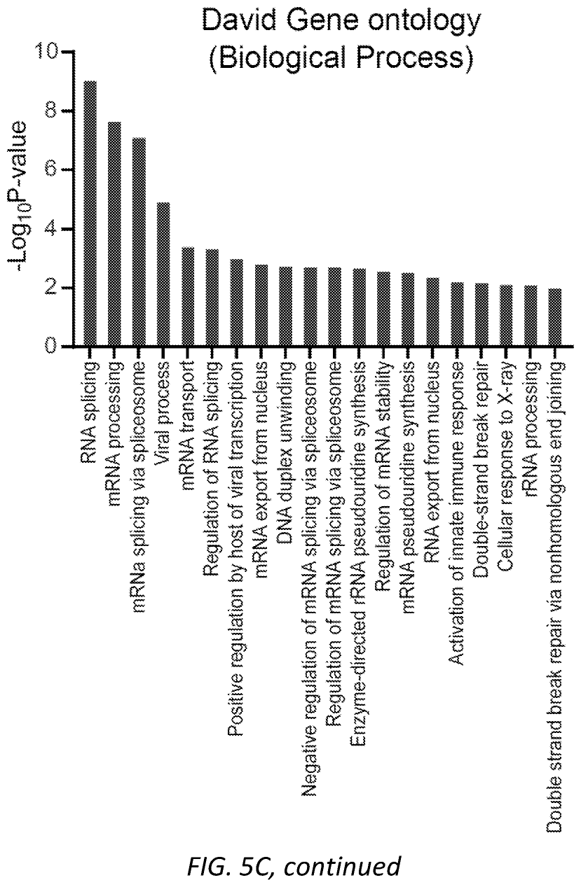

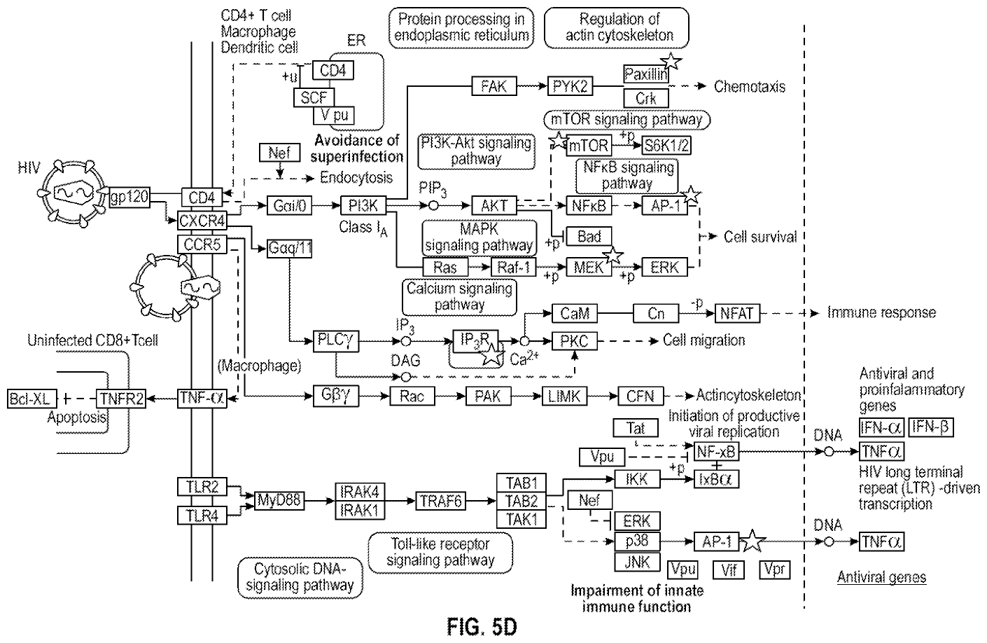

To further gain insight into the mechanism of acquisition of drug resistance in QM and EMT plasticity, we performed phospho-proteomics analysis with QM and E PDAC cells following 2 weeks of FOLFIRINOX treatment (FIG. 5A). Volcano plots represent up and downregulated phosphopeptides in QM and E respectively. As shown in FIGS. 5B-C, QM PDAC cell lines actively respond to FOLFIRINOX treatment and have significant various changes in roughly 900 phospho-peptides, as opposed to the E cell line PDAC 6, which had few differentially phosphorylated proteins. Analysis of the up-regulated phosphosites in QM PDACs demonstrated enrichment in proteins related to regulation of spliceosome and viral processes (FIG. 5C). Based on these data, we identified several potential targets and pathways by which Oxo may be blocking EMT within our cell lines in FIG. 5D.

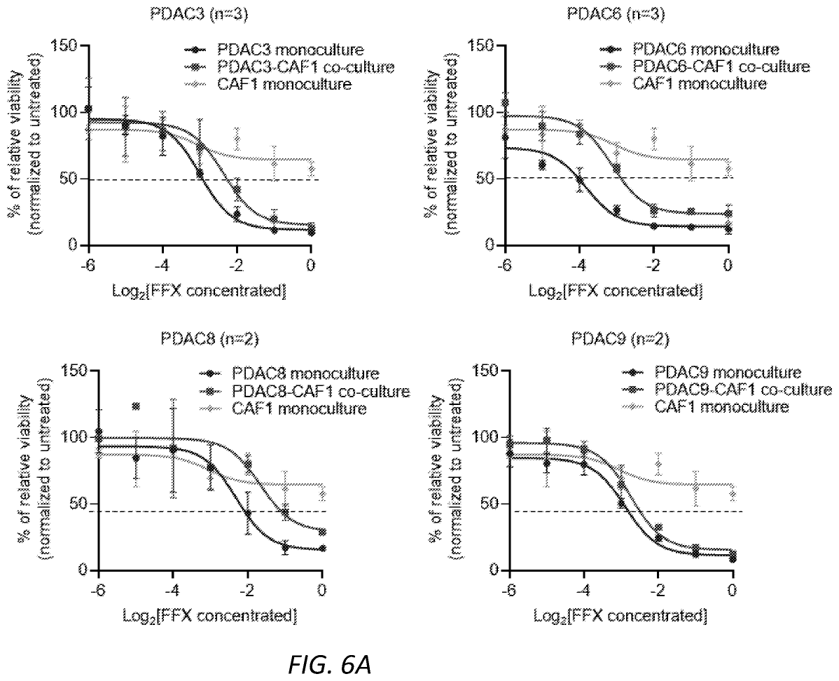

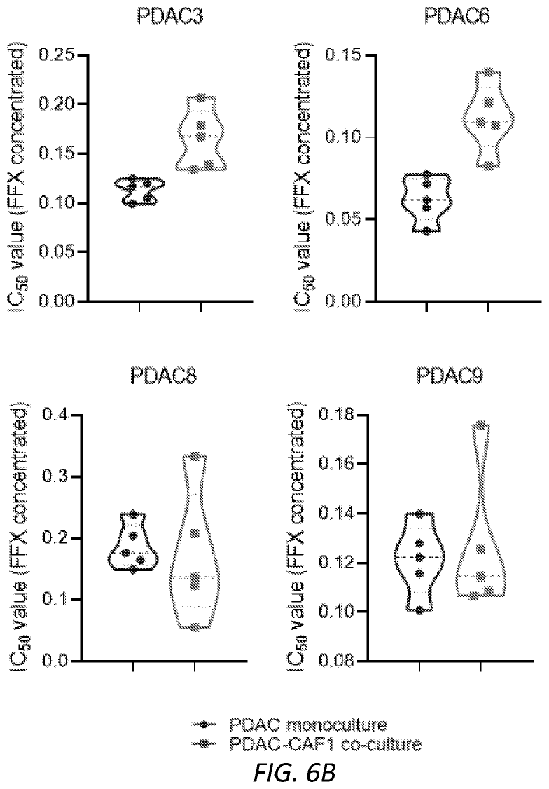

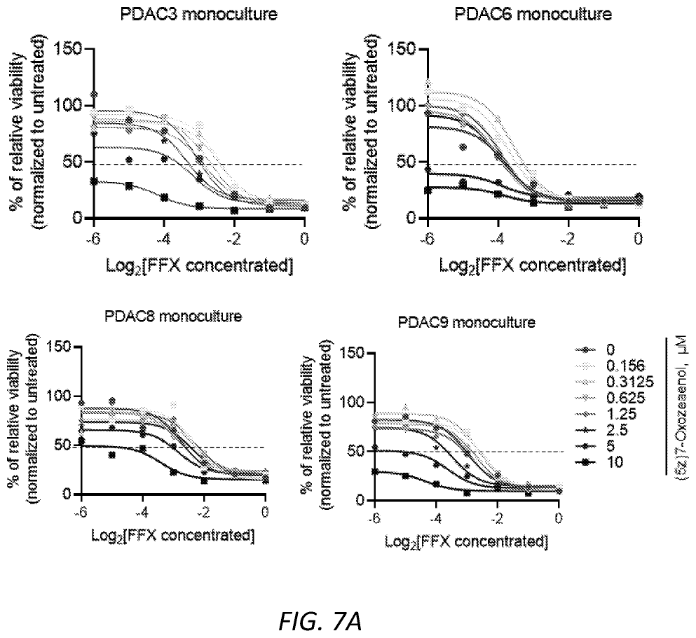

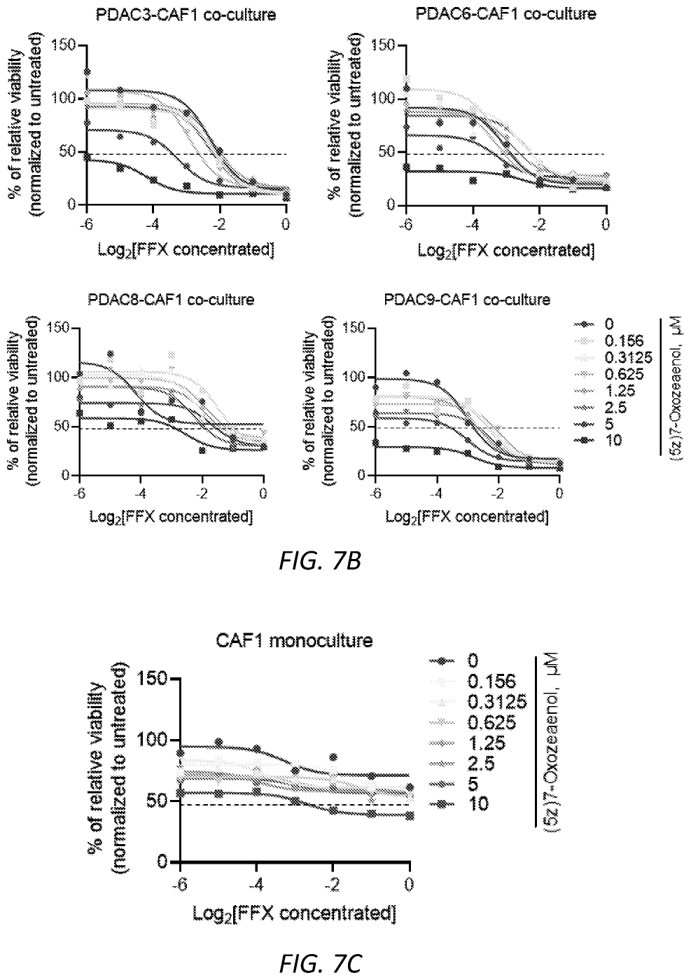

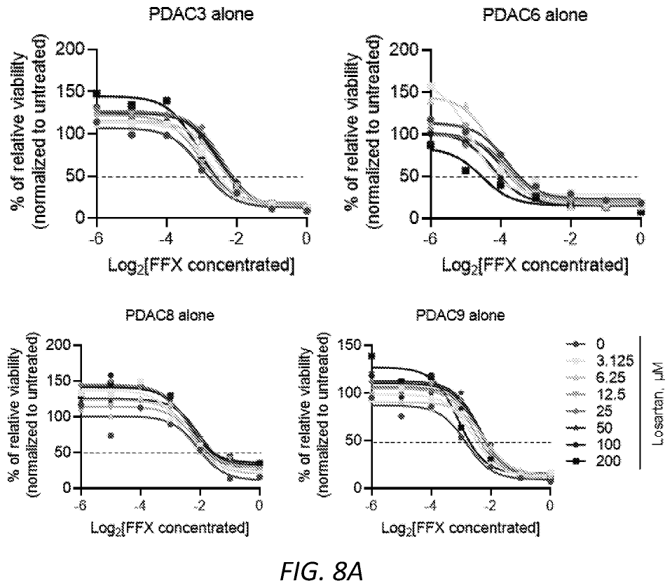

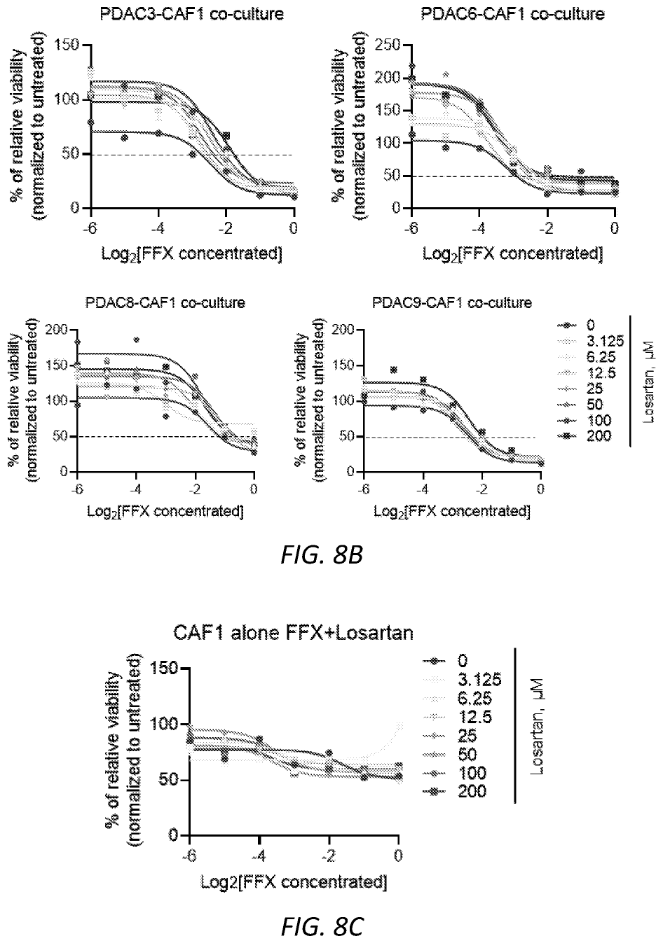

PDAC cells were co-cultured with fibroblasts (CAF) to more closely mimic the tumor microenvironment. As shown in FIGS. 6A-B, PDAC-CAF co-culture desensitized the PDAC cells to FOLFIRINOX treatment versus monoculture particularly in PDAC3 and PDAC6 co-cultures. Treatment with Oxo sensitized cells to FOLFIRINOX treatment, in both monocultures and CAF co-culture conditions (FIGS. 7A-B), while having little effect on CAFs alone (FIG. 7C). This was in contrast to treatment with Losartan, which did not affect PDAC cell line response to FOLFIRINOX in either mono- or co-cultures (FIGS. 8A-B) and which also had no effect on CAFs alone (FIG. 8C).

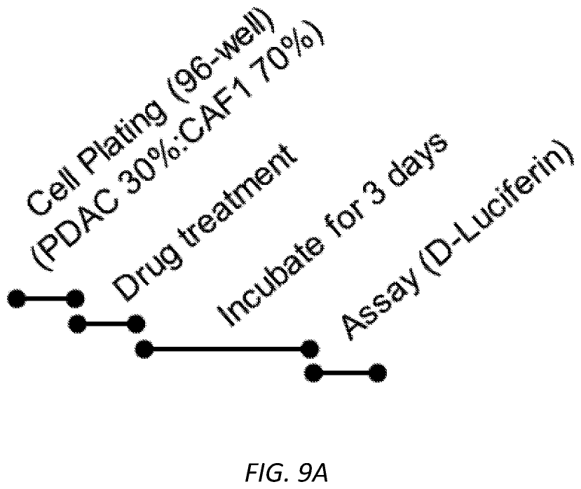

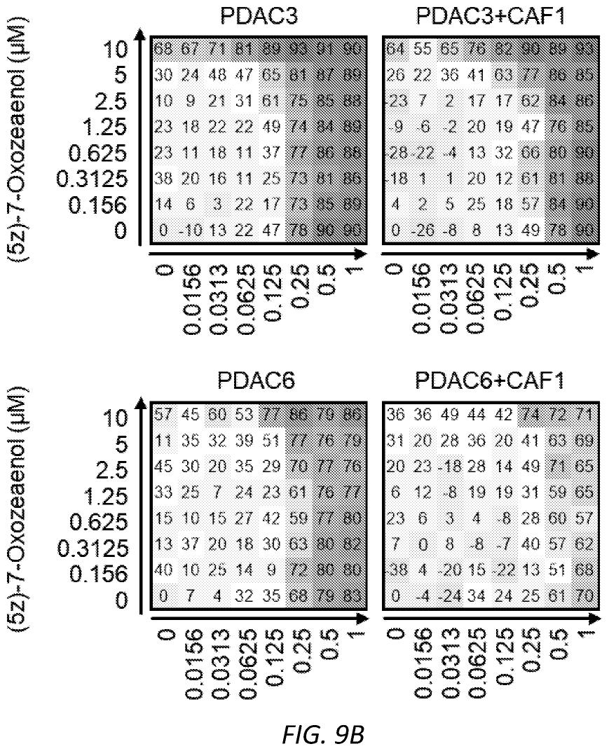

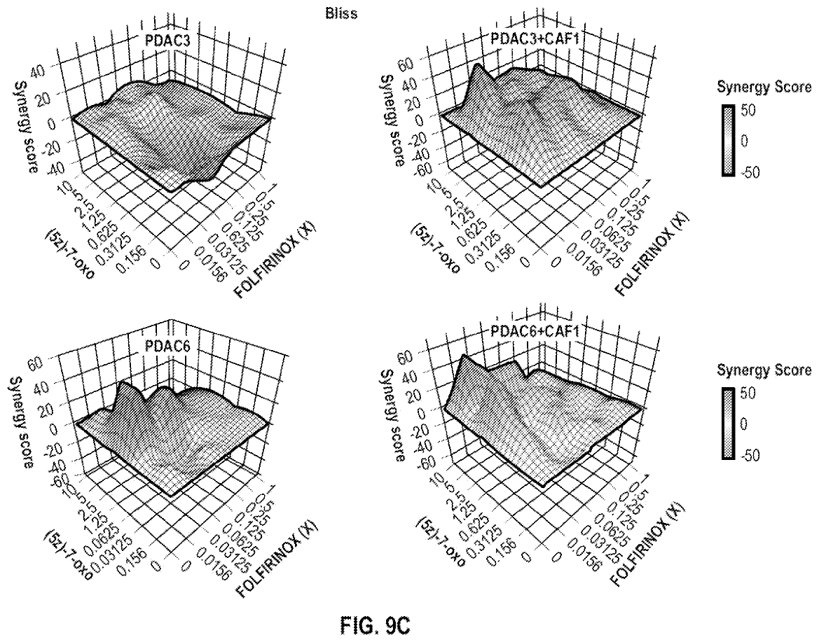

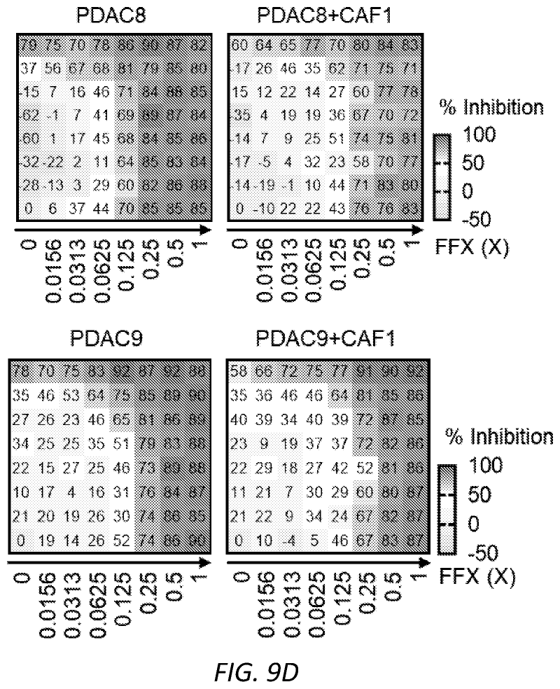

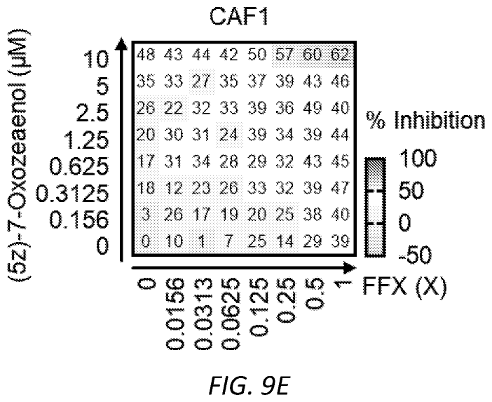

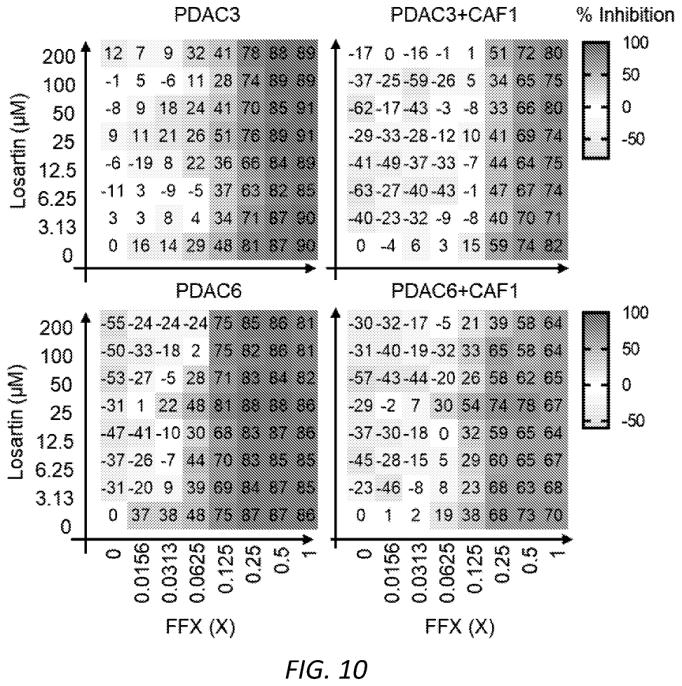

In order to evaluate whether oxo could synergize with FOLFIRINOX, we performed combinatorial drug treatment in PDAC monoculture and PDAC/CAF co-culture settings (FIG. 9A). PDAC monoculture and co-cultures were treated with increasing doses of both Oxo and FOLFIRINOX, and cell viability was measured after 3 days, with both Oxo and FOLFIRINOX demonstrating dose-dependent cell killing in both monoculture and co-culture settings (FIGS. 9B-D). In order to assess for synergistic activity between Oxo and FOLFIRINOX, we used the Bliss independence model to analyze our drug combination experiments (Bliss, 1939; Yadav et al., 2015). Bliss synergy testing demonstrated that the Oxo and FOLFIRINOX combination showed significant synergy in both PDAC monoculture and co-culture settings (FIG. 9C-D). This was in contrast to CAF monoculture, in which Oxo and FOLFIRINOX did not demonstrate nearly as much cell killing (FIG. 9E). This was also in contrast to the combination of Losartan and FOLFIRINOX, for which Losartan did not demonstrate any additional killing when combined with FOLFIRINOX, and instead led to increased cell growth in low FOLFIRINOX settings (FIG. 10). Thus, the combination of Oxo and FOLFIRINOX leads to synergistic killing of PDAC cells, suggesting this treatment as a novel regimen for treatment of patient PDAC.

These results showed that QM cell lines (PDAC3, PDAC9) are resistant to FFX treatment and demonstrate higher migration and invasion in comparison to E cell line (PDAC6). (5z)-7-Oxozeaenol treatment in both PDAC subtypes decreased QM signature concomitantly with E enrichment. Moreover, (5z)-7-Oxozeaenol reduced migration and invasion in QM PDACs, and synergistically increased their sensitivity to FFX. Neither genetic ablation nor selective inhibition (with Takinib) of TAK1 succeeded to phenocopy the (5z)-7-Oxozeaenol effect across PDAC cell lines. Phospho-proteomic analysis suggests that (5z)-7-Oxozeaenol might be targeting other potential proteins involved in the regulation of spliceosome and viral processes. Combination studies with (5z)-7-Oxozeaenol and FOLFIRINOX demonstrate synergistic effects, potentially due to the promotion of the epithelial state by (5z)-7-Oxozeaenol, which has previously been shown to be more susceptible to FOLFIRINOX treatment. Overall, these data demonstrate that (5z)-7-Oxozeaenol and FOLFIRINOX represents a treatment regimen for increased killing of PDAC tumor cells.

### REFERENCES

Bliss, Ann. Appl. Biol. 26, 585-615 (1939).
Boivin et al., WO 2003/076424.
Collisson et al. Nat. Med. (17):500-503 (2011).
Ellestad et al., Chirality. 31:110-117 (2019).
Iriondo et al., Nat Commun. 9: 1994 (2018).
Joo et al., Biomaterials. 34(12): 3098-3109 (2013).
Kim and Choi, Kidney Res Clin Prac. 31(2):94-105 (2012).
Ligoro et al., Cell. 178(1):160-175.e27 (2019).
Lim and Grierson, US20230087078
Porter al., PNAS. 116(52):26835-26845 (2019).
Singh et al., Cell. Feb 17;148(4):639-50 (2012).
Wang et al., Theranostics. 12(2): 657-674 (2022).
Yadav et al., Comp & Struct Biot J. 13: 504-513 (2015).
Yu et al., Nature. 487(7408): 510-513 (2012).
Zhang et al., Clin Cancer Res. 23(7): 1748-1759 (2017).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a carcinoma in a subject, the method comprising administering to the subject a therapeutically effective amount of (5z)-7-oxozeaenol (Oxo) or an analog thereof, optionally in combination with chemotherapy.

2. The method of claim 1, wherein the carcinoma is an adenocarcinoma.

3. The method of claim 2, wherein the adenocarcinoma is pancreatic adenocarcinoma or colorectal adenocarcinoma.

4. The method of claim 1, wherein the Oxo or analog thereof is administered in a nanoparticle.

5. The method of claim 1, wherein the method comprises administering Oxo.

6. The method of claim 1, wherein the method comprises administering E6201 (1H-2-Benzoxacyclotetradecin-1,7(8H)-dione, 14-(ethylamino)-3,4,9,10-tetrahydro-8,9,16-trihydroxy-3,4-dimethyl-, (3S,4R,5Z,8S,9S,1lE)-); L-783,277; hypothemycin; ER-803064; or an exo-enone analogue of (5Z)-7-oxozeaenol.

7. The method of claim 1, wherein the chemotherapy comprises administering one, two, three, or all four of, leucovorin calcium (folinic acid), fluorouracil, irinotecan hydrochloride, and oxaliplatin.

8. The method of claim 7, wherein the chemotherapy comprises administering a FOLFIRINOX, FOLFOX, FOLFIRI, or FOLFOXIRI chemotherapy regimen.

9-16. (canceled)

17. A method of treating a carcinoma in a subject, the method comprising administering to the subject a therapeutically effective amount of (5z)-7-oxozeaenol (Oxo) or an analog thereof, in combination with chemotherapy comprising administering a FOLFIRINOX, FOLFOX, FOLFIRI, or FOLFOXIRI chemotherapy regimen.

18. The method of claim 17, wherein the carcinoma is an adenocarcinoma.

19. The method of claim 18, wherein the adenocarcinoma is pancreatic adenocarcinoma or colorectal adenocarcinoma.

20-22. (canceled)
